SEQUENCE LISTING (30 sequences)

SEQ ID NO: 1 - protein, 20 aa, Artificial Sequence
  Description of Artificial Sequence Synthetic peptide
FPRPGGGGNGDFEEIPEEYL

SEQ ID NO: 2 - protein, 20 aa, Artificial Sequence
  Description of Artificial Sequence Synthetic peptide
FPRPGGGGNGDFEEIPEEYL

SEQ ID NO: 3 - protein, 4 aa, Artificial Sequence
  Description of Artificial Sequence Synthetic peptide
GGGG

SEQ ID NO: 4 - protein, 5 aa, Artificial Sequence
  Description of Artificial Sequence Synthetic peptide
PGGGG

SEQ ID NO: 5 - protein, 4 aa, Artificial Sequence
  Description of Artificial Sequence Synthetic peptide
PGGG

SEQ ID NO: 6 - protein, 6 aa, Artificial Sequence
  Description of Artificial Sequence Synthetic peptide
RPGGGG

SEQ ID NO: 7 - protein, 5 aa, Artificial Sequence
  Description of Artificial Sequence Synthetic peptide
RPGGG

SEQ ID NO: 8 - protein, 4 aa, Artificial Sequence
  Description of Artificial Sequence Synthetic peptide
RPGG

SEQ ID NO: 9 - protein, 7 aa, Artificial Sequence
  Description of Artificial Sequence Synthetic peptide
PRPGGGG

SEQ ID NO: 10 - protein, 6 aa, Artificial Sequence
  Description of Artificial Sequence Synthetic peptide
PRPGGG

SEQ ID NO: 11 - protein, 5 aa, Artificial Sequence
  Description of Artificial Sequence Synthetic peptide
PRPGG

SEQ ID NO: 12 - protein, 4 aa, Artificial Sequence
  Description of Artificial Sequence Synthetic peptide
PRPG

SEQ ID NO: 13 - protein, 5 aa, Artificial Sequence
  Description of Artificial Sequence Synthetic peptide
FPRPG

SEQ ID NO: 14 - protein, 6 aa, Artificial Sequence
  Description of Artificial Sequence Synthetic peptide
GGGGNG

SEQ ID NO: 15 - protein, 7 aa, Artificial Sequence
  Description of Artificial Sequence Synthetic peptide
PGGGGNG

SEQ ID NO: 16 - protein, 8 aa, Artificial Sequence
  Description of Artificial Sequence Synthetic peptide
RPGGGGNG

SEQ ID NO: 17 - protein, 9 aa, Artificial Sequence
  Description of Artificial Sequence Synthetic peptide
PRPGGGGNG

SEQ ID NO: 18 - protein, 5 aa, Artificial Sequence
  Description of Artificial Sequence Synthetic peptide
GGGNG

SEQ ID NO: 19 - protein, 4 aa, Artificial Sequence
  Description of Artificial Sequence Synthetic peptide
GGNG

SEQ ID NO: 20 - protein, 4 aa, Artificial Sequence
  Description of Artificial Sequence Synthetic peptide
GGGG

SEQ ID NO: 21 - protein, 4 aa, Artificial Sequence
  Description of Artificial Sequence Synthetic peptide
NGDF

SEQ ID NO: 22 - protein, 10 aa, Artificial Sequence
  Description of Artificial Sequence Synthetic peptide
DFEEIPEEYL

SEQ ID NO: 23 - protein, 12 aa, Artificial Sequence
  Description of Artificial Sequence Synthetic peptide
NGDFEEIPEEYL

SEQ ID NO: 24 - protein, 16 aa, Artificial Sequence
  Description of Artificial Sequence Synthetic peptide
GGGGNGDFEEIPEEYL

SEQ ID NO: 25 - protein, 14 aa, Artificial Sequence
  Description of Artificial Sequence Synthetic peptide
GGNGDFEEIPEEYL

SEQ ID NO: 26 - protein, 4 aa, Artificial Sequence
  Description of Artificial Sequence Synthetic peptide
NGDF

SEQ ID NO: 27 - protein, 10 aa, Artificial Sequence
  Description of Artificial Sequence Synthetic peptide
DFEEIPEEYL

SEQ ID NO: 28 - protein, 16 aa, Artificial Sequence
  Description of Artificial Sequence Synthetic peptide
GGGGNGDFEEIPEEYL

SEQ ID NO: 29 - protein, 4 aa, Artificial Sequence
  Description of Artificial Sequence Synthetic peptide
GGGG

SEQ ID NO: 30 - protein, 10 aa, Artificial Sequence
  Description of Artificial Sequence Synthetic peptide
DFEEIPEEYL

(19) United States
(12) Patent Application Publication
(10) Pub. No.: US 2017/0029467 A1
(43) Pub. Date: Feb. 2, 2017

(54) METHOD OF PRODUCING BIVALIRUDIN

(71) Applicant: Ambiopharm, Inc., North Augusta, SC (US)

(72) Inventors: Guoqing Zhang, Shanghai (CN); Ruoping Zhang, North August, SC (US); Juncai Bai, North Augusta, SC (US)

(21) Appl. No.: 15/168,745

(22) Filed: May 31, 2016

Related U.S. Application Data
(60) Provisional application No. 62/199,085, filed on Jul. 30, 2015.

(51) Int. Cl. C07K 7/08; C07K 14/815

(52) U.S. Cl. CPC C07K 7/08; C07K 14/815

(57) ABSTRACT

The present disclosure provides a method of producing bivalirudin using a peptide fragment or peptide fragments on solid phase peptide synthesis that minimizes, or eliminates, the production of bivalirudin molecules having too few or too many glycine residues.

### SEQUENCE LISTING

The instant application contains a Sequence Listing which has been submitted electronically in ASCII format and is hereby incorporated by reference in its entirety. Said ASCII copy, created on Aug. 15, 2016, is named 47399-501001US_SL.txt and is 6,906 bytes in size.

### TECHNICAL FIELD

The present disclosure relates to methods for producing bivalirudin. More particularly, the present disclosure relates to methods for producing bivalirudin using a peptide fragment on solid phase peptide synthesis process that substantially reduces or eliminates impurities.

### BACKGROUND

Solid phase peptide synthesis techniques were a breakthrough for producing peptides, first invented by R. Bruce Merrifield. Typically, solid phase peptide synthesis involves attaching a first amino acid whose amino group is protected by a protecting group to a solid phase support, removing the protecting group of the first amino acid with a de-protective agent, activating a carboxyl of a protected second amino acid with an activating group, such as N,N′-dicyclohexyl carbodiimide (DCC), and reacting the first amino acid with the second amino acid to yield a protected dipeptide on the solid phase support. By repeating these steps, a peptide chain may be extended from the carboxyl terminus (C-terminus) to the amino terminus (N-terminus). Once a peptide chain of a desired length is obtained, the protecting group of the N-terminus amino acid is removed, and the peptide chain may be released from the solid phase support or resin by hydrolyzing the bond (e.g. an ester bond) between the first amino acid and the solid phase support with a strong acid such as, for example, hydrofluoric acid (HF). In this manner, a peptide of interest may be obtained.

Thrombin inhibitors are considered to be promising anti-thrombosis drugs. For example, bivalirudin, an anticoagulant peptide, is a bivalent hirudin (hirulog) that has been shown to be therapeutically effective for inhibiting thrombin. Hirudin can be extracted from a blood-sucking leech, i.e., Hirudo medicinalis.

Existing methods for producing bivalirudin using solid phase peptide synthesis techniques are not very practical. For example, these methods typically use a process that couples one amino acid derivative at a time sequentially, which produces peptides that contain many impurities. Disadvantageously, such methods typically produce bivalirudin that is contaminated with impurities that include glycine-deletion and/or glycine addition peptide forms. Unfortunately, these glycine-deletion and/or glycine addition peptide forms pose a significant challenge to the production of bivalirudin because they are very difficult to remove by standard chromatographic purification processes. Accordingly, there is an urgent need for cost efficient methods of producing high purity bivalirudin using solid phase peptide synthesis techniques.

### SUMMARY OF THE DISCLOSURE

The present disclosure relates to methods for producing bivalirudin. More particularly, the present disclosure relates to methods for producing bivalirudin using a peptide fragment or fragments on a solid phase peptide synthesis process that substantially reduces or eliminates impurities.

In one aspect, the present disclosure provides a method for producing bivalirudin using a peptide fragment or peptide fragments on a solid phase peptide synthesis (SPPS), that may include the steps of initiating SPPS of bivalirudin with a protecting group (PG)-Leu-Resin; sequentially coupling in a C-terminal to N-terminal direction, one or more PG-amino acids and one or more PG-peptide fragments to produce bivalirudin (SEQ ID NO. 2), where the one or more PG-peptide fragments include at least one glycine (Gly) residue.

In an embodiment, the at least one PG-peptide fragment may be Asn(Trt)-Gly and the PG may be Boc or Fmoc.

In an embodiment, the at least one PG-peptide fragment may be Gly-Gly-Gly-Gly (SEQ ID NO: 3) and the PG may be Boc or Fmoc.

In an embodiment, the at least one PG-peptide fragment may be Gly-Gly-Gly and the PG may be Boc or Fmoc.

In an embodiment, the at least one PG-peptide fragment may be Gly-Gly and the PG may be Boc or Fmoc.

In an embodiment, the at least one PG-peptide fragment may be Pro-Gly-Gly-Gly-Gly (SEQ ID NO: 4), Pro-Gly-Gly-Gly (SEQ ID NO: 5), Pro-Gly-Gly, or Pro-Gly and the PG may be Boc or Fmoc.

In an embodiment, the at least one PG-peptide fragment may be Arg(Pbf)-Pro-Gly-Gly-Gly-Gly (SEQ ID NO: 6), Arg(Pbf)-Pro-Gly-Gly-Gly (SEQ ID NO: 7), Arg(Pbf)-Pro-Gly-Gly (SEQ ID NO: 8), or Arg(Pbf)-Pro-Gly and the PG may be Boc or Fmoc.

In an embodiment, the at least one PG-peptide fragment may be Pro-Arg(Pbf)-Pro-Gly-Gly-Gly-Gly (SEQ ID NO: 9), Pro-Arg(Pbf)-Pro-Gly-Gly-Gly (SEQ ID NO: 10), Pro-Arg(Pbf)-Pro-Gly-Gly (SEQ ID NO: 11), or Pro-Arg(Pbf)-Pro-Gly (SEQ ID NO: 12) and the PG may be Boc or Fmoc.

In an embodiment, the at least one PG-peptide fragment may be D-Phe-Pro-Arg(Pbf)-Pro-Gly-Gly-Gly-Gly, D-Phe-Pro-Arg(Pbf)-Pro-Gly-Gly-Gly, D-Phe-Pro-Arg(Pbf)-Pro-Gly-Gly, or Phe-Pro-Arg(Pbf)-Pro-Gly (SEQ ID NO: 13) and the PG may be Boc or Fmoc.

In an embodiment, the at least one PG-peptide fragment may be Gly-Gly-Gly-Gly-Asn(Trt)-Gly (SEQ ID NO: 14), Pro-Gly-Gly-Gly-Gly-Asn(Trt)-Gly (SEQ ID NO: 15), Arg(Pbf)-Pro-Gly-Gly-Gly-Gly-Asn(Trt)-Gly (SEQ ID NO: 16), Pro-Arg(Pbf)-Pro-Gly-Gly-Gly-Gly-Asn(Trt)-Gly (SEQ ID NO: 17), or D-Phe-Pro-Arg(Pbf)-Pro-Gly-Gly-Gly-Gly-Asn(Trt)-Gly and the PG may be Boc or Fmoc.

In an embodiment, the at least one PG-peptide fragment may be Fmoc-Gly-Gly-Gly-Asn(Trt)-Gly (SEQ ID NO: 18).

In an embodiment, the at least one PG-peptide fragment may be Fmoc-Gly-Gly-Asn(Trt)-Gly (SEQ ID NO: 19).

In an embodiment, the at least one PG-peptide fragment may be Fmoc-Gly-Asn(Trt)-Gly.

In an embodiment, the resin may be Wang resin.

In an embodiment, the at least one PG-peptide fragment may be Fmoc-Asn(Trt)-Gly and Fmoc-Gly-Gly-Gly-Gly (SEQ ID NO: 20).

In an embodiment, the at least one PG-peptide fragment may be Fmoc-Asn(Trt)-Gly and Fmoc-Gly-Gly.

In an embodiment, the method may further include the steps of cleaving the bivalirudin from the resin; and purifying the cleaved bivalirudin by high pressure liquid chromatography (HPLC).

In an embodiment, the bivalirudin may include an amount of a plus-Gly and/or a minus-Gly impurity of less than about 1.0%.

In an embodiment, the bivalirudin may include an amount of a plus-Gly and/or a minus-Gly impurity of less than about 0.5%.

In an embodiment, the bivalirudin may include an amount of a plus-Gly and/or a minus-Gly impurity of less than about 0.25%.

In an embodiment, the bivalirudin may include an amount of a plus-Gly and/or a minus-Gly impurity of less than about 0.10%.

In an aspect, the present disclosure provides a method for producing bivalirudin using a peptide fragment or peptide fragments on solid phase peptide synthesis (SPPS), that may include attaching a first protected leucine (Leu) to a resin; de-protecting the protected Leu; reacting a second protected amino acid with the de-protected Leu to form a peptide bond there between, repeating de-protecting and reacting amino acids of 11-19 residues of SEQ ID NO 2 sequentially in a C-terminal to N-terminal direction; de-protecting the protected Asp(OtBu); reacting a protected Asn(Trt)-Gly with the de-protected Asp(OtBu); de-protecting the Asn(Trt); reacting a protected Gly-Gly-Gly-Gly (SEQ ID NO: 3) with the de-protected Asn(Trt); repeating de-protecting and reacting amino acids of 1-4 residues of SEQ ID NO 2, sequentially, and de-protecting the last amino acid residue, cleaving the bivalirudin from the resin.

In an embodiment, the protecting group may be Fmoc or Boc.

In an embodiment, the resin may be Wang resin.

In an embodiment, a de-protective agent may be used in de-protecting the amino acids, and the de-protective agent may include an amount of about 3 to 20% of piperidine and an amount of about 0.5 to 10% of bicyclic amidine, based on the total volume thereof.

In an embodiment, a condensing agent may be used for reacting the amino acids to form the peptide bonds, and the condensing agent may be selected from the group consisting of N,N′-diisopropyl carbodiimide, O-(7-aza-benzotriazole-1-yl)-N,N,N′,N′-tetramethyl uronium hexafluoro phosphate, O-(benzotriazole-1-yl)-N,N,N,N-4-methyl-uronium tetrafluoroborate/N-methyl morpholine, (benzo triazol-1-yl-O)tripyrrolidine phosphonium hexafluorophosphate, 1-hydroxy benzotriazole, and a mixture thereof.

In an embodiment, the step of repeating de-protecting and reacting amino acids of 1-4 residues of SEQ ID NO 2, sequentially, and de-protecting the last amino acid residue, when a protected Arg(Pbf) is reacted, pentafluorophenol may be used to condense the protected-Arg(Pbf)-OH with the peptide bound to the resin.

In an embodiment, the step of repeating de-protecting and reacting amino acids of 1-4 residues of SEQ ID NO 2, sequentially, and de-protecting the last amino acid residue, when a protected Arg(HCl) is reacted, pentafluorophenol may be used to condense the protected-Arg(Pbf)-OH with the peptide bound to the resin.

In an embodiment, a cleaving agent may be used in cleaving the bivalirudin from the resin, and the cleaving agent may be trifluoroacetic acid, triisopropyl silane, and water, with a volume ratio thereof 95-60:5-10:5-30.

In an embodiment, the cleaved peptide may be precipitated.

In an embodiment, the bivalirudin may include an amount of a plus-Gly and/or a minus-Gly impurity of less than about 1.0%.

In an embodiment, the bivalirudin may include an amount of a plus-Gly and/or a minus-Gly impurity of less than about 0.5%.

In an embodiment, the bivalirudin may include an amount of a plus-Gly and/or a minus-Gly impurity of less than about 0.25%.

In an embodiment, the bivalirudin may include an amount of a plus-Gly and/or a minus-Gly impurity of less than about 0.10%.

In an embodiment, when the PG-peptide fragments include at least two of Gly-Gly, the step of reacting a protected Gly-Gly-Gly-Gly (SEQ ID NO: 3) with the de-protected Asn(Trt) may further include the steps of reacting a first protected Gly-Gly with the de-protected Asn(Trt); de-protecting the Gly, and reacting a second protected Gly-Gly with the de-protected Gly.

In an aspect, the present disclosure may further include a pharmaceutical composition that includes bivalirudin produced by any of the above methods.

In an embodiment, the bivalirudin may include an impurity of plus-Gly and/or minus-Gly less than about 1.0%.

### DEFINITIONS

The term “plus-Gly impurity”, as used herein, may indicate a peptide by-product produced during the solid phase synthesis of bivalirudin, which includes at least one, at least two, or at least three or more of additional Gly residues relative to the bivalirudin amino acid sequence (SEQ ID NO. 2). Such unintended plus-Gly impurity (also referred to as “glycine-addition”) may occur at, or near, the Gly residues normally found in SEQ ID NO. 2.

The term “minus-Gly impurity”, as used herein, may indicate a peptide by-product produced during the solid phase synthesis of bivalirudin, which deletes at least one, at least two, or at least three or more Gly residues relative to the bivalirudin amino acid sequence (SEQ ID NO. 2). Such unintended minus-Gly impurity (also referred to as “glycine-deletion”) may occur near or at the Gly residues in SEQ ID NO. 2.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

### BRIEF DESCRIPTION OF THE DRAWINGS

The above and other features of the present invention will now be described in detail with reference to certain exemplary embodiments thereof illustrated in the accompanying drawings which are given herein below by way of illustration only, and thus are not limitative of the present invention, and wherein:

FIG. 1 shows exemplary high performance liquid chromatography (HPLC) traces of crude bivalirudin preparations prepared by solid phase peptide synthesis according to an exemplary embodiment of the present disclosure. In particular, a crude bivalirudin preparation prepared by adding 20 amino acids sequentially is compared to a crude bivalirudin preparation prepared by coupling a 4-Gly fragment (SEQ ID NO: 3) to the peptide-resin according to an exemplary embodiment of the disclosure.

FIG. 2 show exemplary high performance liquid chromatography (HPLC) traces of purified bivalirudin preparations prepared by solid phase peptide synthesis according to an exemplary embodiment of the present disclosure. In particular, a final product of bivalirudin prepared by adding 20 amino acids sequentially is compared to a final product bivalirudin prepared by incorporating a 4-Gly fragment (SEQ ID NO: 3) into the solid phase peptide synthesis.

### DETAILED DESCRIPTION OF THE DISCLOSURE

The present disclosure features methods for producing bivalirudin. More particularly, the present disclosure relates to methods for producing bivalirudin using a peptide fragment on a solid phase peptide synthesis process that substantially reduces or eliminates impurities. The present disclosure is based, at least in part, on the unexpected discovery that incorporating protected peptide fragments such as, for example, Fmoc-Asn(Trt)-Gly, Fmoc-Gly-Gly-Gly-Gly (SEQ ID NO: 20), and/or Fmoc-Gly-Gly into the solid phase peptide synthesis (SPPS) of bivalirudin greatly reduced the occurrence of plus-Gly and/or minus-Gly impurities in the resulting bivalirudin preparation.

Advantages of the disclosure are summarized below:

1) SPPS techniques as described herein are very cost-efficient compared with conventional methods for producing bivalirudin, and generally reduce the cost of bivalirudin synthesis by about 50%;

2) SPPS techniques according to the disclosure provide bivalirudin preparations having high purity: the purity of the crude bivalirudin of the disclosure resulting from this synthesis process can be 77-93%, and the glycine-deletion and glycine-addition impurities closely eluted with the main peak can be controlled to less than about 0.10% after a preparative HPLC purification step to meet the pharmaceutical requirements;

3) The techniques disclosed herein significantly decrease the risk associate with bivalirudin synthesis because methyl tert-butyl ether (MTBE) can be used instead of ether, which improves the production safety level. For example, ether is an extremely flammable chemical with flash point of −45° C., and boiling point of 34.6° C., meanwhile methyl tert-butyl ether has a flash point of −28° C. and a boiling point of 55.3° C.; and

4) The techniques disclosed herein are environmentally-friendly because the method is a solid phase synthesis process performed without water, which allows organic solvents for washing to be recycled.

### Solid Phase Peptide Synthesis

A peptide can be synthesized naturally via ribosome or non-ribosomal biosynthesis pathways in a living cell, or chemically synthesized using a synthesizer. Solid phase peptide synthesis generally refers to a chemical synthesis of the peptide using a solid phase support or a resin that retains a peptide or fragment thereof on the surface of the solid phase support or the resin. In particular, the peptide can be manipulated and produced efficiently by using solid phase peptide synthesis. For instance, a bivalirudin comprising 20 amino acids can be manufactured for its therapeutic use with substantially improved yield and purity.

In one aspect, the present disclosure provides a solid phase peptide synthesis and reagents used for the same. The solid phase peptide synthesis may comprise: attaching a first amino acid to a resin; protecting amino acids and de-protecting amino acids; condensing (e.g., coupling) amino acids by forming a peptide bond; and cleaving the produced peptide chain from the resin. In particular, processes of protecting, de-protecting, and condensing may be repeated sequentially until the desired length of the peptide is made.

In a preferred aspect, in the solid phase peptide synthesis, each amino acid may be protected at amino groups thereof. Particularly, the protecting group (PG) may protect an amine of each amino acid that is used as a building block of the peptide (e.g. bivalirudin), and any protecting groups for amino group well-known to those of ordinary skill in the art can be used.

In preferred embodiments, the protecting group used for the solid phase synthesis may be at least one protecting group selected from the group consisting of a carbobenzyloxy (Cbz) group, a p-Methoxybenzyl carbonyl (Moz or MeOZ) group, a tert-Butyloxycarbonyl (Boc) group, a 9-Fluorenylmethyloxycarbonyl (Fmoc) group, an acetyl (Ac) group, a benzoyl (Bz) group, a benzyl (Bn) group, a carbamate group, a p-Methoxybenzyl (PMB), a 3,4-Dimethoxybenzyl (DMPM) group, a p-methoxyphenyl (PMP) group, a tosyl (Ts) group, and a sulfonamides group.

In particular embodiments, the protecting group for solid phase synthesis of bivalirudin may be Fmoc or Boc.

In a preferred aspect, in solid phase peptide synthesis, the protected amino acids are de-protected using a chemical agent, i.e. de-protective agent. As such, the protected amino acid is de-protected using the de-protective agent to remove the protecting group and expose the amino group for subsequent coupling or peptide bond formation, and any de-protecting agents well-known to those of ordinary skill in the art may be used.

In preferred embodiments, the de-protective agent used for the solid phase synthesis of a peptide (e.g., bivalirudin) may be selected from the group consisting of piperidine, bicyclic amidine (DBU), 1-hydroxy benzotriazole (HOBt), 3-hydroxy-1,2,3-benzo triazine-4(3H)-one (HOOBt), dimethylformamide (DMF), and any mixtures thereof.

In particular embodiments, the de-protective agent used for the solid phase synthesis of bivalirudin may comprise piperidine and bicyclic amidine (DBU). In other embodiments, the de-protective agent may further comprise 1-hydroxy benzotriazole (HOBt) and 3-hydroxy-1,2,3-benzo triazine-4(3H)-one (HOOBt).

In certain embodiments, the de-protective agent may comprise an amount of about 3 to 20% of piperidine, and an amount of about 0.5 to 10% of bicyclic amidine (DBU), based on the total volume of the de-protecting agent. In certain embodiments, the de-protective agent may further comprise an amount of about 0 to 20% of 1-hydroxy benzotriazole (HOBt), or an amount of about 0 to 10% of 3-hydroxy-1,2,3-benzo triazine-4(3H)-one (HOOBt), based on the total volume of the de-protecting agent.

Alternatively, in certain embodiments, the de-protective agent may comprise between 5 and 15% of piperidine and between 1 and 7% of bicyclic amidine (DBU) based on the total volume of the de-protecting agent. The de-protective agent may further comprise an amount of about 0.5 to 10% of 1-hydroxy benzotriazole (HOBt), an amount of about 0.2 and 5% of 3-hydroxy-1,2,3-benzo triazine-4(3H)-one (HOOBt), or a mixture thereof, based on the total volume of the de-protecting agent.

Further, in certain embodiments, the de-protective agent may comprise an amount of about 3 to 20% of piperidine and an amount of about 0.5 to 10% of bicyclic amidine (DBU), based on the total volume of the de-protecting agent. The de-protective agent may further comprise an amount of about 0 and 20% of 1-hydroxy benzotriazole (HOBt), an amount of about 0 and 10% of 3-hydroxy-1,2,3-benzo triazine-4(3H)-one (HOOBt), or a mixture thereof, based on the total volume of the de-protecting agent.

According to an exemplary embodiment for the synthesis of bivalirudin using solid phase peptide synthesis, when DBU is used as the de-protective agent, purity of the synthesized peptide (bivalirudin) may be substantially improved. For example, in HPLC trace analysis of the final bivalirudin product, peaks immediately adjacent (i.e., before and/or after) to the main peak (i.e., bivalirudin product) may disappear completely.

Furthermore, according to an exemplary embodiment for the synthesis of bivalirudin that includes amino acid residues of Asn-Gly, or Asn(Trt)-Gly, a de-protective agent comprising piperidine, DBU, HOBt, HOOBT, or a mixture thereof may be particularly effective.

In a preferred aspect, solid phase peptide synthesis of bivalirudin according to the disclosure may implement a condensing (coupling) agent to promote or facilitate formation of a peptide bond between the amino group and the carboxyl group from consecutive amino acids in the peptide. It is contemplated within the scope of the disclosure that any condensing agent well-known in the art may be used.

In particular embodiments, the condensing agent may be, but is not limited to, carbodiimide, ByPOB, HATU, and TBTU. Further, the condensing agent may be selected from the group consisting of N,N′-diisopropyl carbodiimide (DIC), O-(7-aza-benzotriazole-1-yl)-N,N,N′,N′-tetramethyl uronium hexafluoro phosphate (HATU), O-(benzotriazole-1-yl)-N,N,N,N-4-methyl-uronium tetrafluoroborate (TBTU), (benzo triazol-1-yl-O)tripyrrolidine phosphonium hexafluorophosphate (PyBOP), 1-hydroxy benzotriazole (HOBt), N-methyl morpholine (NMM), or a mixture thereof.

According to an exemplary embodiment of the disclosure, a condensing agent comprising HOBt/DIC or TBTU/NMM may be used at each condensation step. In particular, for steps of condensing arginine (Arg) or Arg(Pbf), pentafluorophenol may be used to reduce the production cost and reduce impurity due to arginine deletion. With respect to condensing Arg or Arg(Pbf), an amount of 1.5-6.0 equivalents of PG-Arg(Pbf)-OH or Fmoc-Arg(Pbf) relative to the resin, pentafluorophenol in an amount of about 1.5-6.0 equivalents to the resin may be used as the condensing agent, and the resin linked to the peptide chain may be mixed for about 12 to 36 hrs.

In a preferred aspect of the solid phase peptide synthesis, a cleavage agent may be used to release condensed or coupled amino acids, or peptide from the resin or the solid phase support. Any cleavage agent well-known to those of ordinary skill in the art may be used without limitation. In preferred embodiments, the cleavage agent for separating full length bivalirudin from the resin may a weak acid solution. In particular embodiments, the cleavage agent may comprise TFA and HCl in aqueous solution.

In another aspect, the present disclosure provides a method for producing bivalirudin using solid phase peptide synthesis. In Table 1, SEQ ID NO. 1 represents an amino acid sequence of bivalirudin being attached to a resin and SEQ ID NO. 2 represent an amino acid sequence of bivalirudin.

TABLE 1

| | |
|---|---|
| SEQ ID NO: 1 | PG-_{D}-Phe¹-Pro²-Arg(Pbf)³-Pro⁴-Gly⁵- |
| | Gly⁶-Gly⁷-Gly⁸-Asn(Trt)⁹-Gly¹⁰-Asp |
| | (OtBu)¹¹-Phe¹²-Glu(OtBu)¹³-Glu |
| | (OtBu)¹⁴-Ile¹⁵-Pro¹⁶-Glu(OtBu)¹⁷- |
| | Glu(OtBu)¹⁸-Tyr(tBu)¹⁹-Leu²⁰-Resin |
| SEQ ID NO: 2 | _{D}-Phe¹-Pro²-Arg(Pbf)³-Pro⁴-Gly⁵- |
| | Gly⁶-Gly⁷-Gly⁸-Asn(Trt)⁹-Gly¹⁰- |
| | Asp(OtBu)¹¹-Phe¹²-Glu(OtBu)¹³- |
| | Glu(OtBu)¹⁴-Ile¹⁵-Pro¹⁶-Glu |
| | (OtBu)¹⁷-Glu(OtBu)¹⁸-Tyr(tBu)¹⁹- |
| | Leu²⁰ |

In an exemplary embodiment, the solid phase peptide synthesis may comprise the following steps:

a) loading a protection group (PG)-Leu-OH onto a resin;

b) removing the PG- with a de-protective agent;

c) condensing another PG-amino acid with the amino acid bound to the resin; and

d) cleaving the peptide from the resin to yield the bivalirudin of SEQ ID NO. 2.

Preferably, in step a), the resin may include any resin well-known to those of ordinary skill in the art. As used herein, the resin may support or retain an amino acid or a peptide by forming a bond (e.g. covalent bond) with the functional group of peptide terminus, such as a carboxyl group of the amino acid. The resin may typically include a polymer core and a linker group between the C-terminal amino acid and the polymer core. The resin, particularly the polymer core, is physically stable and chemically inert with respect to the reagents used during the peptide synthesis. The resin also allows contact and attachment of an amino acid (i.e. a first amino acid). In addition, the linker group may be suitably inert during the peptide synthesis, but once the peptide synthesis is terminated, the bond between the linker group and the amino acid may be cleaved with the cleaving agent without deteriorating the peptide, such that the cleavage properties of the resin can be modified by permanently attaching suitable linkers. By manipulating the structure of the linker, the resins that can be used in the solid phase peptide synthesis may vary as desired.

In a preferred embodiment, the resin may be a Wang resin, a Merrifield resin, a PAM resin, a hydroxymethyl resin, a cross linked-polystyrene resin, a polyacrylamide resin, and the like.

In particular embodiments, the resin may be a Wang resin. In particular, a Wang resin having a substitution rate of 0.40-1.4 mmol/g may be used.

In a preferred embodiment, the protecting group in steps a) and c) may be at least one selected from the group consisting of carbobenzyloxy (Cbz) group, p-Methoxybenzyl carbonyl (Moz or MeOZ) group, tert-Butyloxycarbonyl (Boc) group, 9-Fluorenylmethyloxycarbonyl (Fmoc) group, acetyl (Ac) group, benzoyl (Bz) group, benzyl (Bn) group, carbamate group, p-Methoxybenzyl (PMB), 3,4-Dimethoxybenzyl (DMPM) group, p-methoxyphenyl (PMP) group, tosyl (Ts) group, and sulfonamides group.

Preferably, in steps a) and c), the protecting group (PG) for solid phase synthesis of bivalirudin may be Fmoc or Boc.

In particular embodiments, in step a), the PG may be Fmoc. Furthermore, an amount of about 1.8 to about 3.0 resin equivalent of Fmoc-Leu-OH may be reacted with the Wang resin.

Preferably, in step b), the PG (e.g. Fmoc) may be removed with the de-protective agent. Any de-protecting method used in solid phase peptide synthesis in the related art may be used without limitation. For example, the protected amino-acid resin may be washed with the de-protective agent.

In a preferred embodiment, the de-protective agent used for the solid phase synthesis of bivalirudin may be at least one selected from the group consisting of piperidine, bicyclic amidine (DBU), 1-hydroxy benzotriazole (HOBt), 3-hydroxy-1,2,3-benzo triazine-4(3H)-one (HOOBt), and dimethylformamide (DMF).

In particular, the de-protective agent may comprise piperidine and bicyclic amidine (DBU), and in particular embodiments, the de-protective agent may further comprise 1-hydroxy benzotriazole (HOBt) and 3-hydroxy-1,2,3-benzo triazine-4(3H)-one (HOOBt).

In preferred embodiments, in step c), protected second or additional amino acids may be added to the amino acid-resin mixture. In particular embodiments, an amount of about 1.5 to 4.5 resin equivalent of PG-amino acid (e.g. Fmoc amino acid) may be used.

Furthermore, in step c), prior to adding the PG amino-acid to the resin mixture, the protected amino acid may be de-protected with an amount of about 1.5 to 3.0 resin equivalents of HOBt. The de-protected amino acid may be suitably dissolved in a solvent, for example, DMF. Any suitable solvent known in the art may be used. The amount of solvent used may be based on the amount of resin. For example, 1 mL of the solvent per each g of the resin may be used.

In a preferred embodiment, when the mixture is added to the resin mixture in step c), the condensing agent may be added to the resin mixture to react the carboxyl group of the added amino acid and the amino group on the resin peptide to form peptide bonds there between.

In another preferred embodiment, the condensing agent for producing the bivalirudin may include at least one agent selected from the group consisting of DIC, PyBOP, HATU, TBTU, HOBt, NMM, and a mixture thereof.

In particular embodiments, the condensing agent comprising HOBt/DIC or TBTU/NMM may be used at each condensation step. In particular, an amount of about 2.0 to 6.0 resin equivalent of DIC or TBTU may be added.

In preferred aspects, in step c), peptide forming condensation reaction with the condensing agent may be performed for about 10 minutes, 20 minutes, 30 minutes, 40 minutes, 50 minutes, 60 minutes, 70 minutes, 80 minutes, 90 minutes, 100 minutes, 110 minutes, 120 minutes, or greater. In certain embodiments, the condensing reaction may be performed for about 90 min.

In preferred aspects, the reaction after condensation (peptide forming) may be suitably diluted with a solvent, such as DMF, to a volume of about 2 mL/g resin, 3 mL/g resin, 4 mL/g resin, 5 mL/g resin, 6 mL/g resin, 7 mL/g resin, 8 mL/g resin, 9 mL/g resin, 10 mL/g resin, or greater. In certain embodiments, the reaction may be suitably diluted with DMF to a volume of about 4 mL/g resin.

In preferred embodiments, in step c), the reaction may be cooled to a temperature of about 20° C., about 19° C., about 18° C., about 17° C., about 16° C., about 15° C., about 14° C., about 13° C., about 12° C., about 12° C., about 10° C., or less. In certain embodiments, the reaction may be cooled to a temperature of about 10° C.

Preferably, the reaction after condensing in step c) may be stored for about 1 hour, about 2 hours, about 3 hours, about 4 hours, about 5 hours, about 6 hours, about 7 hours, about 8 hours, about 9 hours, about 10 hours, or greater, or particularly for about 6 hrs.

In certain embodiments, in step c), when the amino acid added to the resin is Fmoc-Arg(Pbf)-OH for the third residue from the N-terminus of bivalirudin, the condensation reaction may be performed as follows: an amount of about 1.5 to 6.0 equivalents of Fmoc-Arg(Pbf)-OH and pentafluorophenol are dissolved with DMF or KSCN (3 mL/g resin), and then an amount of about 1.5 to 6.0 equivalents of a condensing agent such as DIC, HATU, TBTU, or PyBOP are added and stirred for about 90 min. The resultant Fmoc-Arg(Pbf)-OPfp/DMF solution is added to the resin and stirred for about 12-36 hrs.

In preferred embodiments, in step d), the peptide chain may be separated or cleaved from the resin by using a cleavage agent. In particular embodiments, the cleavage agent may be selected from the group consisting of trifluoroacetic acid (TFA), triisopropyl silane (TIS), and water. In certain embodiments, the cleavage agent may be a mixture of TFA, TIS, and water at a volume ratio of about 95-60:5-10:5-30.

### Solid Phase Peptide Synthesis of Bivalirudin

The present disclosure provides a method for producing bivalirudin having amino acids of SEQ ID NO. 2. In particular, the bivalirudin may be synthesized on a solid phase support or resin in the form of SEQ ID NO: 1, and then separated or cleaved from the resin as a crude compound.

In a preferred embodiment, the bivalirudin of SEQ ID NO. 2 may be synthesized by using solid phase supports as described above. That is, each protected amino acid of the bivalirudin may be sequentially attached or coupled in a direction from the C-terminus to N-terminus. Accordingly, the protecting, de-protecting, and coupling reactions may be repeated sequentially for at least about 20 times or more.

In other preferred aspect, the method of producing bivalirudin comprises coupling peptides or peptide fragments that comprise at least two amino acids or more.

In preferred embodiments, the peptide fragments may comprise at least two, at least three, at least four, at least five, at least six, at least seven, at least eight, at least nine, at least ten or more amino acids. Further in preferred embodiments, the peptide fragments may comprise any portion of the bivalirudin peptide residues shown in, for example, SEQ ID NO. 2. In particular, the peptide fragments may comprise consecutive or sequential amino acids from SEQ ID NO. 2.

In particular embodiments, the peptide fragments may be prepared to include at least one Gly. In certain embodiments, the peptide fragments may be at least Gly-Gly, at least Gly-Gly-Gly-, at least Gly-Gly-Gly-Gly (SEQ ID NO: 3), or more. In other certain embodiments, the peptide fragments may include at least Asn(Trt)-Gly, at least Asn(Trt)-Gly-Asp(OtBu), at least Asn(Trt)-Gly-Asp-Phe (SEQ ID NO: 21), or more.

In preferred embodiments, the peptide fragments may be prepared to include the protecting group at an amino group of the N-terminus. Furthermore, the peptide fragments may be protected with a protecting group such as, for example, Boc and Fmoc.

In preferred embodiments, a first peptide fragment may be coupled to the peptide chain formed on the solid-support or resin, and a second peptide fragment may be coupled (condensed) to the peptide chain formed on the solid phase support or resin.

In particular embodiments, the first peptide fragment may include amino acids of Asn-Gly, or Asn(Trt)-Gly that are protected with Fmoc or Boc. More particularly, the first peptide fragment may be Fmoc-Asn(Trt)-Gly. For example, the Fmoc-Asn(Trt)-Gly may be coupled to the peptide chain on the resin, i.e. Asp(OtBu)-Phe-Glu(OtBu)-Glu(OtBu)-Ile-Pro-Glu(OtBu)-Glu(OtBu)-Tyr(tBu)-Leu-resin (SEQ ID NO: 22), which may be treated with the de-protective agent, to form a peptide Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23).

Further, in particular embodiments, the second peptide fragments may include amino acids of Gly-Gly-Gly-Gly (SEQ ID NO: 3) that is protected by Boc or Fmoc, and more particularly, Fmoc-Gly-Gly-Gly-Gly (SEQ ID NO: 20). For example, the Fmoc-Gly-Gly-Gly-Gly (SEQ ID NO: 20) is coupled to the peptide chain on the resin, i.e. Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23), which is treated with the de-protective agent, to form a peptide Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 24).

Moreover, each amino acid of 1-4 residues of bivalirudin may be attached to the above prepared peptide-resin. Particularly, Fmoc-Pro-OH, Fmoc-Arg(Pbf)-OH, Fmoc-Pro-OH and Fmoc-_{D}-Phe-OH are sequentially coupled (condensed) to the peptide resins, thereby producing SEQ ID NO. 1. The prepared peptide is further de-protected and cleaved from the resin to producing the bivalirudin of SEQ ID NO. 2.

In another preferred aspect, a first peptide fragment, a second peptide fragment, and a third peptide fragment may be coupled (condensed) sequentially, and this coupled peptide may be further coupled (condensed) in the peptide chain formed on the solid phase support or resin.

In particular embodiments, the first peptide fragment may include amino acids of Asn-Gly, or Asn(Trt)-Gly that are protected with Fmoc or Boc, and more particularly, the first peptide fragment may be Fmoc-Asn(Trt)-Gly. For example, the Fmoc-Asn(Trt)-Gly may be coupled to the peptide chain on the resin, i.e. Asp(OtBu)-Phe-Glu(OtBu)-Glu(OtBu)-Ile-Pro-Glu(OtBu)-Glu(OtBu)-Tyr(tBu)-Leu-resin (SEQ ID NO: 22), which is treated with the de-protective agent, to form a peptide Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23).

In further embodiments, the second peptide fragments may have amino acids of Gly-Gly that is protected by Boc or Fmoc, more particularly, Fmoc-Gly-Gly. For example, the Fmoc-Gly-Gly may be coupled to the peptide chain on the resin, i.e. Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23), which is treated with the de-protective agent, to form a peptide Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 25).

Subsequently, in particular embodiments, the second peptide fragments may have amino acids of Gly-Gly that are protected by Boc or Fmoc, and more particularly, Fmoc-Gly-Gly. For example, the Fmoc-Gly-Gly may be coupled to the peptide chain on the resin, i.e. Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 25), which may be treated with the de-protective agent, to form a peptide Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 24).

Moreover, each amino acid of 1-4 residues of bivalirudin may be attached to the above prepared peptide-resin. Particularly, Fmoc-Pro, Fmoc-Arg(Pbf), Fmoc-Pro and Fmoc-_{D}-Phe may be sequentially coupled (condensed) to the peptide resins, thereby producing SEQ ID NO. 1. The prepared peptide may be further de-protected and cleaved from the resin to producing the bivalirudin of SEQ ID NO. 2.

In an exemplary embodiment, the method may include the steps of:

a) attaching a first protected leucine (Leu) to a resin;

b) de-protecting the protected Leu;

c) reacting a second protected amino acid with the de-protected Leu to form a peptide bond therebetween,

d) repeating de-protecting and reacting amino acids of 11-19 residues of SEQ ID NO 2 sequentially;

e) de-protecting the protected Asp(OtBu);

f) reacting a protected Asn(Trt)-Gly with the de-protected Asp(OtBu);

g) de-protecting the Asn(Trt);

h) reacting a protected Gly-Gly-Gly-Gly (SEQ ID NO: 3) with the de-protected Asn(Trt);

i) repeating de-protecting and reacting amino acids of 1-4 residues of SEQ ID NO 2, sequentially, and de-protecting the last amino acid residue,

g) cleaving the bivalirudin from the resin.

Alternatively, in an exemplary embodiment, the method may include the following steps of h-1)-h-3) replacing the step of h) above, when the peptide fragments includes at least two of Gly-Gly:

h-1) reacting a first protected Gly-Gly with the de-protected Asn(Trt);

h-2) de-protecting the Gly,

h-3) reacting a second protected Gly-Gly with the de-protected Gly.

The protecting group may be Fmoc or Boc.

The resin may be Wang resin.

The de-protective agent may be used in de-protecting the amino acids, and the de-protective agent comprises an amount of about 3 to 20% of piperidine and an amount of about 0.5 to 10% of bicyclic amidine, based on the total volume thereof.

The condensing agent may be used for reacting the amino acids to form the peptide bonds, and the condensing agent is selected from the group consisting of N,N′-diisopropyl carbodiimide, O-(7-aza-benzotriazole-1-yl)-N,N,N′,N′-tetramethyl uronium hexafluoro phosphate, O-(benzotriazole-1-yl)-N,N,N,N-4-methyl-uronium tetrafluoroborate/N-methyl morpholine, (benzo triazol-1-yl-O)tripyrrolidine phosphonium hexafluorophosphate, 1-hydroxy benzotriazole, and a mixture thereof.

The cleaving agent may be used in cleaving the bivalirudin from the resin, and the cleaving agent is trifluoroacetic acid, triisopropyl silane, and water, with a volume ratio thereof of about 95-60:5-10:5-30.

The cleaved crude bivalirudin of SEQ ID NO. 2 may be suitably processed and precipitated.

For example, the obtained (crude) bivalirudin may be mixed with MTBE or ether to yield a peptide precipitate. Particularly, the MTBE or ether may be cooled to about −10 to 0° C., e.g. by an ice-water bath or a refrigerant known to those of ordinary skill in the art, and the precipitate may be suitably washed with the ether other than the ether used for precipitation and separated by filtration or centrifugation.

Alternatively, the crude bivalirudin may be purified using preparative HPLC purification to meet the pharmaceutical impurity requirements.

Preferably, the purity of the resultant bivalirudin may be 90%, 91%, 92%, 93%, 94%, 95%, 96%, 97%, 98%, 99% or greater.

In preferred embodiments, the bivalirudin produced by the method of the present disclosure may include substantially reduced amount of impurities, particularly a plus-Gly impurity, a minus-Gly impurity, or mixtures thereof.

Preferably, the bivalirudin of the pharmaceutical composition may include a plus-Gly impurity, a minus-Gly impurity, or mixture thereof, in less than about 1.00%, less than about 0.90%, less than about 0.80%, less than about 0.70%, less than about 0.60%, less than about 0.50%, less than about 0.45%, less than about 0.40%, less than about 0.35%, less than about 0.30%, less than about 0.25%, less than about 0.20%, less than about 0.15%, or less than about 0.10%, based on the total area under HPLC trace peaks.

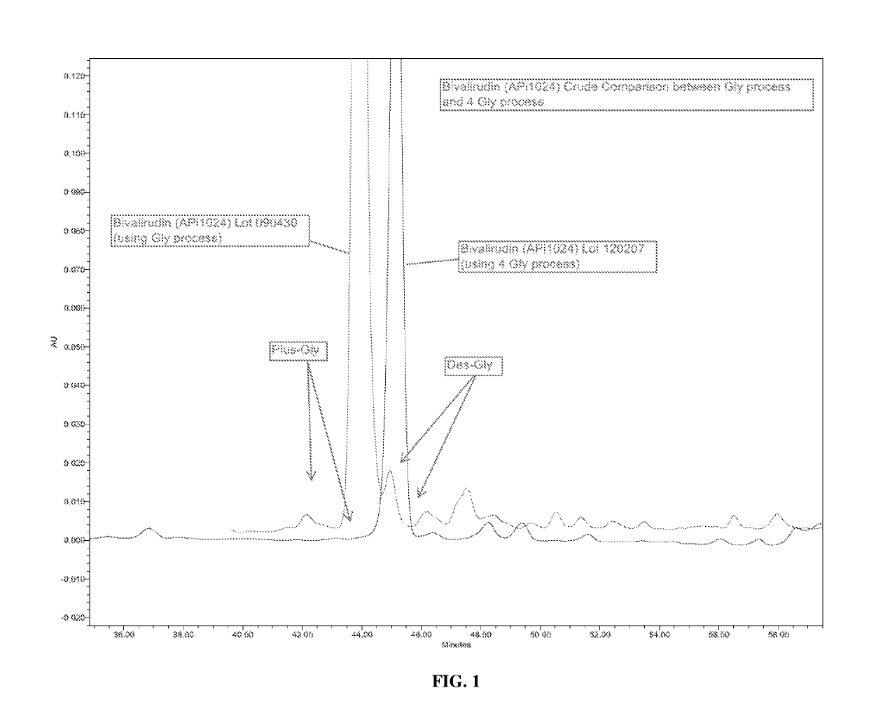

FIG. 1 shows HPLC traces of a crude bivalirudin preparation. The crude bivalirudin (Api1024, LOT 090430) was prepared by using protected amino acids of 1-20 residues as described in Examples 1-2. The crude bivalirudin (Api1024, LOT 120207) was prepared by using protected amino acids of 11-20 residues and sequentially coupling the peptide fragments of Asn(Trt)-Gly and Gly-Gly-Gly-Gly (SEQ ID NO: 3), as described in Examples 3-4.

As shown in FIG. 1, the crude bivalirudin of LOT 120207 included substantially reduced or eliminated plus-Gly or minus-Gly impurities as compared to the crude bivalirudin of LOT 090430.

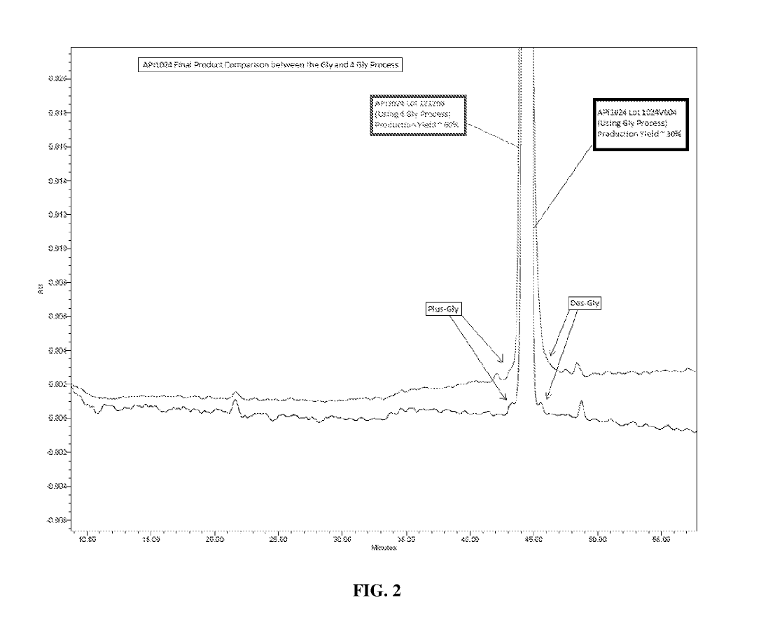

Moreover, FIG. 2 shows HPLC traces of purified bivalirudin as final products. The bivalirudin of LOT 1024V004 was prepared by using protected amino acids of 1-20 residues as described in Examples 1-2. The bivalirudin of LOT 101206 was prepared by using protected amino acids of 11-20 residues and sequentially coupling the peptide fragments of Asn(Trt)-Gly and Gly-Gly-Gly-Gly (SEQ ID NO: 3), as described in Examples 3-4.

As shown in FIG. 1, the final product bivalirudin of LOT 101206 included substantially reduced or eliminated plus-Gly or minus-Gly impurities as compared to the bivalirudin of LOT 1024V004. As such, according to preferred embodiments of the present disclosure, bivalirudin for therapeutic or pharmaceutical use can be produced with substantially improved purity and yield.

### Pharmaceutical Compositions

In one embodiment, further provided is a pharmaceutical composition that comprises bivalirudin and pharmaceutically acceptable salts thereof.

The term “pharmaceutically acceptable salt” as used herein may refer to any type of salts used in a medicinal composition or a pharmaceutical composition, which are well-known to those of ordinary skill in the art. The pharmaceutically acceptable salt may be selected suitably such that the salts may not affect or deteriorate the therapeutic effect of the bivalirudin, particularly when it is administered together with the bivalirudin. Exemplary salts may include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids may also be used. Such pharmaceutically acceptable salts also can include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

In preferred pharmaceutical compositions of the disclosure, the bivalirudin may be prepared by using solid phase synthesis.

In preferred embodiments, the bivalirudin may be synthesized by a method comprising: coupling peptides or peptide fragments that comprise at least two amino acids or more.

For example, the peptide fragments may comprise at least two, at least three, at least four, at least five, at least six, at least seven, at least eight, at least nine, or at least ten or more amino acids. Further in preferred embodiments, the peptide fragments may comprise any portion of the bivalirudin peptide residues as shown in SEQ ID NO. 2, in particular, the peptide fragments may comprise consecutive or sequential amino acids from SEQ ID NO. 2.

In particular embodiments, the peptide fragments may be prepared to include at least one Gly. In certain embodiments, the peptide fragments may be at least Gly-Gly, at least Gly-Gly-Gly-, at least Gly-Gly-Gly-Gly (SEQ ID NO: 3) or more. In other certain embodiments, the peptide fragments may be at least Asn-Gly, at least Asn-Gly-Asp, at least Asn-Gly-Asp-Phe (SEQ ID NO: 26) or more.

In preferred embodiments, the peptide fragments may be prepared to include the protecting group at an amino group of the N-terminus. The peptide fragments may be further protected with the protecting group, such as Boc and Fmoc.

In preferred embodiments, a first peptide fragment may be coupled to the peptide chain formed on the solid-support or resin, and a second peptide fragment may be coupled (condensed) to the peptide chain formed on the solid phase support or resin.

In particular embodiments, the first peptide fragment may have amino acids of Asn-Gly, or Asn(Trt)-Gly that is protected with Fmoc or Boc, more particularly, the first peptide fragment may be Fmoc-Asn(Trt)-Gly. For example, the Fmoc-Asn(Trt)-Gly is coupled to the peptide chain on the resin, i.e. Asp(OrBu)-Phe-Glu(OtBu)-Glu(OtBu)-Ile-Pro-Glu(OtBu)-Glu(OtBu)-Tyr(tBu)-Leu-resin (SEQ ID NO: 27), which is treated with the de-protective agent, to form a peptide Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23).

Further, in particular embodiments, the second peptide fragments may have amino acids of Gly-Gly-Gly-Gly (SEQ ID NO: 3) that is protected by Boc or Fmoc, more particularly, Fmoc-Gly-Gly-Gly-Gly (SEQ ID NO: 20). For example, the Fmoc-Gly-Gly-Gly-Gly (SEQ ID NO: 20) is coupled to the peptide chain on the resin, i.e. Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23), which is treated with the de-protective agent, to form a peptide Gly-Gly-Gly-Gly-Asn(Trt)-Gly-Asp(OtBu)-Phe-Glu(OtBu-Glu(OtBu)-Ile-Pro-Glu(OtBu)-Glu(OtBu)-Tyr(tBu)-Leu-resin (SEQ ID NO: 28).

Moreover, each amino acid of 1-4 residues of bivalirudin may be attached to the above prepared peptide-resin. Particularly, Fmoc-Pro, Fmoc-Arg(HCl), Fmoc-Pro and Fmoc-_{D}-Phe may be sequentially coupled (condensed) to the peptide resins, thereby producing SEQ ID NO. 1. The prepared peptide is further de-protected and cleaved from the resin to produce the bivalirudin of SEQ ID NO. 2.

In other preferred embodiments, a first peptide fragment, a second peptide fragment, and a third peptide fragment may be coupled (condensed) sequentially, and this coupled peptide may be further coupled (condensed) in the peptide chain formed on the solid phase support or resin.

In particular embodiments, the first peptide fragment may have amino acids of Asn-Gly, or Asn(Trt)-Gly that is protected with Fmoc or Boc, and more particularly, the first peptide fragment may be Fmoc-Asn(Trt)-Gly. For example, the Fmoc-Asn(Trt)-Gly may be coupled to the peptide chain on the resin, i.e. Asp(OtBu)-Phe-Glu(OtBu)-Glu(OtBu)-Ile-Pro-Glu(OtBu)-Glu(OtBu)-Tyr(tBu)-Leu-resin (SEQ ID NO: 22), which is treated with the de-protective agent, to form a peptide Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23).

In embodiments, the second peptide fragments may have amino acids of Gly-Gly that are protected by Boc or Fmoc, more particularly, Fmoc-Gly-Gly. For example, the Fmoc-Gly-Gly may be coupled to the peptide chain on the resin, i.e. Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 23), which is treated with the de-protective agent, to form a peptide Gly-Gly-Asn(Trt)-Gly-Asp(OtBu)-Phe-Glu(OtBu)-Glu(OtBu)-Ile-Pro-Glu(OtBu)-Glu(OtbU)-Tyr(tBu)-Leu-resin (SEQ ID NO: 25).

Subsequently, in particular embodiments, the second peptide fragments may have amino acids of Gly-Gly that is protected by Boc or Fmoc, more particularly, Fmoc-Gly-Gly. For example, the Fmoc-Gly-Gly may be coupled to the peptide chain on the resin, i.e. Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 25), which is treated with the de-protective agent, to form a peptide Gly-Gly-Gly-Gly-Asn(Trt)-Gly-Asp(OtBu)-Phe-Glu(OtBU)-Glu(OtBu)-Ile-Pro-Glu(OtBu)-Glu(OtBu)-Tyr(tBu)-Leu-resin (SEQ ID NO: 24).

Moreover, each amino acid of 1-4 residues of bivalirudin may be attached to the above prepared peptide-resin. Particularly, Fmoc-Pro, Fmoc-Arg(HCl), Fmoc-Pro and Fmoc-_{D}-Phe are sequentially coupled (condensed) to the peptide resins, thereby producing SEQ ID NO. 1. The prepared peptide may be further de-protected and cleaved from the resin to producing the bivalirudin of SEQ ID NO. 2.

As such, the bivalirudin that is prepared as described above may have a substantially reduced impurity ratio, with respect to either plus-Gly impurity or minus-Gly impurity.

In particular embodiments, the bivalirudin of the pharmaceutical composition may include plus-Gly impurity, minus-Gly impurity, or mixture thereof in less than about 1.00%, less than about 0.90%, less than about 0.80%, less than about 0.70%, less than about 0.60%, less than about 0.50%, less than about 0.45%, less than about 0.40%, less than about 0.35%, less than about 0.30%, less than about 0.25%, less than about 0.20%, less than about 0.15%, or less than about 0.10%, based on the total area under HPLC trace peaks.

Further, the purity of the resultant bivalirudin may be 90%, 91%, 92%, 93%, 94%, 95%, 96%, 97%, 98%, 99% or greater.

The following examples are put forth so as to provide those of ordinary skill in the art with a complete disclosure and description of how to make and use the solid phase peptide synthesis methods of the invention, and are not intended to limit the scope of what the inventors regard as their invention.

### EXAMPLES

Unless otherwise specified, the experiments in the following Examples were carried out under normal conditions, or in accordance with the conditions recommended by the manufacturer, and all percentages, ratios, and/or proportions were calculated by weight. Calculating the volume percentage of weights described herein is well-known to those of ordinary skill in the art, e.g., the weight of solute dissolved in 100 mL of solution.

Exemplary chemical agents (and their associated abbreviations) described herein may include, but are not limited to, the following:
Fmoc 9-fluorenylmethoxycarbonyl
Boc Butoxycarbonyl
DMF N,N-dimethylformamide
KSCN Potassium thiocyanate
DBU 1,8-diazabicyclo(5.4.0)undec-7-ene
HOBt 1-hydroxy benzotriazole
DIC N,N′-diisopropyl c+arbodiimide
NMM N-methyl morpholine
Pbf 2,2,4,6,7-5-pentamethyl-benzofuran-5-sulfonyl
Opfp Pentafluorophenyl ester
TFA Trifluoroacetic acid
TIS Triisopropyl silane
MTBE Methyl tert-butyl ether
HOOBT 3-hydroxy-1,2,3-benzo triazine-4(3H)-one
HATU O-(7-aza-benzotriazole-1-yl)-N,N,N′,N′-tetramethyl uranium hexafluoro phosphate
TBTU O-(benzotriazole-1-yl)-N,N,N,N-4-methyl-uronium tetrafluoroborate
PyBOP (benzo triazol-1-yl-O)tripyrrolidine phosphonium hexafluorophosphate

### Example 1

### Preparation of Bivalirudin I

Step 1: Loading Fmoc-Leu-OH to a Resin

2.0 molar equivalents of Fmoc-Leu-OH was activated with 2,6-dichlorobenzoylchloride and pyridine and reacted with Wang resin (having a substitution rate of 0.40-1.4 mmol/g) in a DMF solution.

Step 2: Removing Fmoc

Another DMF solution comprising 15% of piperidine/5% of DBU was added and allowed to react for 30 min so as to remove Fmoc. The resultant resin was washed once with DMF, twice with methanol, and twice with DMF, respectively.

Condensing the Fmoc-amino acid (i.e., the last amino acid was Boc-D-Phe-OH): 1.5-3.0 resin equivalent of Fmoc-amino acid and 1.5 resin equivalent of HOBt were dissolved with DMF (3 mL/g resin); the mixture was added to the resin, and then 3.0 resin equivalents of DIC was added, and allowed to react for 90 min. The whole process was monitored by ninhydrin colorimetric method (e.g., Kaiser). The resultant solution was diluted with DMF at 10° C. to a volume (4 mL/g resin) and then allowed to react for about 6 hrs.

Condensing Fmoc-Arg(Pbf)-OH: 1.5 equivalents of Fmoc-Arg(Pbf)-OH and pentafluorophenol were dissolved with DMF (1 mL/g resin), and then 6.0 equivalents of HATU were added and stirred for 90 min. The resultant Fmoc-Arg(Pbf)-OPfp/DMF solution was added to the resin and stirred for 48 hours at between 5 and 8° C. The whole process was monitored by ninhydrin colorimetric method (e.g., Kaiser).

Washing: After all desired amino acids were condensed, the resin was washed twice with methanol, thrice with DMF, and thrice with methanol, respectively. Subsequently, the resin was dried under vacuum to reach a certain weight and packed, and a yield thereof was calculated according to its weight gain.

Step 3: Preparation of a Cleavage Agent

TFA, TIS, and water with a volume ratio of 80:10:10 were mixed in a vessel to yield a cleavage agent. The cleavage agent was cooled to 0±2° C. by an ice-water bath or a refrigerant.

Cleavage: The peptide resin was slowly added to the cooled cleavage agent. The mixture was stirred for 2-3 hours at less than 5° C. and then was filtered. The resulting filtrate was collected.

Precipitating: MTBE was cooled to −10° C. and the filtrate was added, thereby producing a peptide precipitate. The precipitate was washed thrice with cooled ether. Upon washing, the cooled ether should be sufficient enough to cover the precipitate in the centrifuge tube or in the filter, and the precipitate and the cooled ether were mixed completely by a spatula. The mixture was centrifuged to yield bivalirudin I.

Drying: The solid peptide of bivalirudin I (as shown in Formula II) was transferred to a vessel and dried in a vacuum drying oven or in a dryer at room temperature for more than 6 hrs. Subsequently, the solid peptide was weighed and packed.

The product had purity of 85%. The impurity (retention time of 23.2 min) prior to main peak was less than 1% in content, and the largest single impurity (retention time of 26.3 min) was less than 2.5% in content.

### Example 2

### Preparation of Bivalirudin II

Step 1: Binding Fmoc-Leu to a Resin

3.0 molar equivalents of Fmoc-Leu were activated with 2,6-dichlorobenzoylchloride and pyridine and reacted with Wang resin (having a substitution rate of 1.0-1.2 mmol/g) in a DMF solution. The unreacted groups of the resin were blocked by benzoyl chloride/triethylamine.

Step 2: Removing Fmoc

Five times resin bed volume of DMF solution comprising 10% of piperidine/7% of DBU/3% of HOOBt was added and allowed to react for 30 minutes so as to remove the Fmoc. The resulting resin was washed once with 5 times the resin bed volume of DMF, thrice with 5 times resin bed volume of methanol, and thrice with 5 times resin bed volume of DMF, respectively.

Condensing Fmoc-amino acid (i.e., the last amino acid was Boc-D-Phe-OH): 1.5-2.0 resin equivalent of Fmoc-amino acid and 3.0 resin equivalent of HOBt were dissolved with DMF (5 mL/g resin); the mixture was added to the resin, and then 3.0 resin equivalent of TBTU/NMM was added, and allowed to react for 90 min. The whole process was monitored by ninhydrin colorimetric method (e.g., Kaiser).

Condensing Fmoc-Arg(HCl)—OH: 6.0 equivalents of Fmoc-Arg(HCl)—OH and pentafluorophenol were dissolved with DMF (4 mL/g resin), and then 1.5 equivalents of DIC were added and stirred for 90 min. The resulting Fmoc-Arg(HCl)-OPfp/DMF solution was added to the resin and stirred for 18 hrs.

Washing: After all required amino acids were condensed, the resin was washed twice with 5 times resin bed volume of methanol, thrice with 5 times resin bed volume of DMF, and thrice with 5 times resin bed volume of methanol, respectively. Subsequently, the resin was dried under vacuum to reach a certain weight and packed, and a yield thereof was calculated according to its weight gain.

Step 3: Preparation of a Cutting Agent

TFA, TIS, and water with a volume ratio of 90:5:5 were mixed in a vessel to yield a cutting agent. The cutting agent was cooled to 0±2° C. by an ice-water bath or a refrigerant. The peptide resin was mixed with the cooled cutting agent. The mixture was allowed to react for 2-3 hours at less than 5° C. and then filtered. The resulting filtrate was collected.

Precipitating: To ether cooled to −10° C., the filtrate was added and a peptide precipitate was produced. The precipitate was collected by filtration or centrifugation and washed three times with cooled MTBE. Upon washing, the cooled MTBE covered the precipitate in the centrifuge tube or in the filter, and the precipitate and the cooled MTBE were mixed completely by a spatula. The mixture was centrifuged to yield bivalirudin II.

Drying: The solid peptide of bivalirudin II (as shown in Formula II) was transferred to a vessel and dried in a vacuum drying oven or a dryer at room temperature for more than 6 hrs. Subsequently, the solid peptide was weighed and packed.

The product had purity of 87%. The impurity (retention time of 23.5 min) prior to main peak was 0.63% in content, and the largest single impurity (retention time of 27.1 min) was 2.1% in content.

### Example 3

### Preparation of Bivalirudin III—Using Fmoc-Gly-Gly-Gly-Gly OH (SEQ ID NO: 20) and Fmoc Asn(Trt)-Gly-OH

After obtaining the peptide resin, H-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 30) (prepared as described in Example 1), Fmoc-Asn(Trt)-Gly-OH was introduced to continue the sequence elongation. 1.5-3.0 resin equivalent of Fmoc-Asn(Trt)-Gly-OH and 1.5 resin equivalent of HOBt were dissolved with DMF (3 mL/g resin); the mixture was added to the resin, and then 3.0 resin equivalents of DIC was added, and allowed to react for 90 min. The whole process was monitored by ninhydrin colorimetric method (e.g., Kaiser). The resultant solution was diluted with DMF at 10° C. to a volume (4 mL/g resin) and then allowed to react for about 6 hrs.

The Fmoc was deprotected using the method described in Example 1 and Example 2. Then Fmoc-Gly-Gly-Gly-Gly-OH (SEQ ID NO: 29) was introduced using the method below.

1.5-3.0 resin equivalent of Fmoc-Gly-Gly-Gly-Gly-OH (SEQ ID NO: 29) and 1.5 resin equivalent of HOBt were dissolved with DMSO (1 mL per gram of Fmoc-Gly-Gly-Gly-Gly-OH (SEQ ID NO: 29)) and DMF (4 mL/g resin); the mixture was added to the resin, and then 3.0 resin equivalents of DIC was added, and allowed to react for 90 min. The whole process was monitored by ninhydrin colorimetric method (e.g., Kaiser). The resultant solution was diluted with DMF at 10° C. to a volume (4 mL/g resin) and then allowed to react for about 6 hrs.

The chain elongation was continued as described in previous examples to obtain the Boc-D-Phe-Pro-Arg (Pbf)-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin (SEQ ID NO: 30).

The crude peptide was deprotected and de-attached from the resin as described in previous examples to obtain the Bivalirudin crude peptide.

### Example 4

### Preparation of Bivalirudin III—Using Fmoc-Gly-Gly-OH and Fmoc Asn(Trt)-Gly-OH

After obtaining the peptide resin, H-Asp(OtBu)-Phe-Glu(OtBu)-Glu(OtBu)-Ile-Pro-Glu(OtBu)-Glu(OtBu)-Tyr(tBu)-Leu-resin(prepared as described in Example 1), Fmoc-Asn(Trt)-Gly-OH was introduced to continue the sequence elongation. 1.5-3.0 resin equivalent of Fmoc-Asn(Trt)-Gly-OH and 1.5 resin equivalent of HOBt were dissolved with DMF (3 mL/g resin); the mixture was added to the resin, and then 3.0 resin equivalents of DIC was added, and allowed to react for 90 min. The whole process was monitored by ninhydrin colorimetric method (e.g., Kaiser). The resultant solution was diluted with DMF at 10° C. to a volume (4 mL/g resin) and then allowed to react for about 6 hrs.

The Fmoc was deprotected using the method described in Example 1 and Example 2. Then Fmoc-Gly-Gly-OH was introduced using the method below.

1.5-3.0 resin equivalent of Fmoc-Gly-Gly-OH and 1.5 resin equivalent of HOBt were dissolved with DMSO (1 mL per gram of Fmoc-Gly-Gly-OH) and DMF (4 mL/g resin); the mixture was added to the resin, and then 3.0 resin equivalents of DIC was added, and allowed to react for 90 min. The whole process was monitored by ninhydrin colorimetric method (e.g., Kaiser). The resultant solution was diluted with DMF at 10° C. to a volume (4 mL/g resin) and then allowed to react for about 6 hrs.

Again, the Fmoc was deprotected using the method described in Example 1 and Example 2. The second Fmoc-Gly-Gly-OH was introduced as described above.

The chain elongation was continued as described in previous examples to obtain the Boc-D-Phe-Pro-Arg (Pbf)-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-resin.

The crude peptide was deprotected and de-attached from the resin as described in previous examples to obtain the Bivalirudin crude peptide

### Methods and Materials

The parameters of HPLC of embodiments of the disclosure are listed below:

| | | | | |
|---|---|---|---|---|
| Column | C18 5 u 100 A 250 × | UV detection | 215 | nm |
| | 4.5 mm | wavelength | | |
| Mobile | A: 0.1% TFA aqueous | Velocity of | 1.0 | mL/min |
| phase | solution | flow | | |
| | B: 0.1% TFA acetonitrile | | | |
| | solution | | | |
| Test- | 45° C. | Injection | 5-50 | μL |
| temperature | | volume | | |

Gradient:

| Time (min.) | % A | % B |
|---|---|---|
| 0.0 | 85 | 15 |
| 35.0 | 60 | 45 |
| 35.1 | 20 | 80 |
| 40.0 | 20 | 80 |
| 40.1 | 85 | 15 |
| 45.0 | 85 | 15 |

The retention time of bivalirudin is about 23.7 min and that of main impurity is between 23 and 23.3 min.

While particular embodiments of the disclosure have been shown and described, it will be obvious to those skilled in the art that changes and modifications may be made without departing from the disclosure in its broader aspects, and therefore, the aim in the appended claims is to cover all such changes and modifications as fall within the true spirit and scope of the disclosure.

What is claimed is:

1. A method for producing bivalirudin using a peptide fragment or peptide fragments on solid phase peptide synthesis (SPPS), comprising:
initiating SPPS of bivalirudin with a protecting group (PG)-Leu-Resin; and
sequentially coupling in a C-terminal to N-terminal direction, one or more PG-amino acids and one or more PG-peptide fragments to produce bivalirudin (SEQ ID NO. 2), wherein the one or more PG-peptide fragments include at least one glycine (Gly) residue.

2. The method of claim 1, wherein the at least one PG-peptide fragment is Asn(Trt)-Gly and the PG is Boc or Fmoc.

3. The method of claim 1, wherein the at least one PG-peptide fragment is Gly-Gly-Gly-Gly (SEQ ID NO: 3) and the PG is Boc or Fmoc.

4. The method of claim 1, wherein the at least one PG-peptide fragment is Gly-Gly-Gly and the PG is Boc or Fmoc.

5. The method of claim 1, wherein the at least one PG-peptide fragment is Gly-Gly and the PG is Boc or Fmoc.

6. The method of claim 1, wherein the at least one PG-peptide fragment is Pro-Gly-Gly-Gly-Gly (SEQ ID NO: 4), Pro-Gly-Gly-Gly (SEQ ID NO: 5), Pro-Gly-Gly, or Pro-Gly and the PG is Boc or Fmoc.

7. The method of claim 1, wherein the at least one PG-peptide fragment is Arg(Pbf)-Pro-Gly-Gly-Gly-Gly (SEQ ID NO: 6), Arg(Pbf)-Pro-Gly-Gly-Gly (SEQ ID NO: 7), Arg(Pbf)-Pro-Gly-Gly (SEQ ID NO: 8), or Arg(Pbf)-Pro-Gly and the PG is Boc or Fmoc.

8. The method of claim 1, wherein the at least one PG-peptide fragment is Pro-Arg(Pbf)-Pro-Gly-Gly-Gly-Gly (SEQ ID NO: 9), Pro-Arg(Pbf)-Pro-Gly-Gly-Gly (SEQ ID NO: 10), Pro-Arg(Pbf)-Pro-Gly-Gly (SEQ ID NO: 11), or Pro-Arg(Pbf)-Pro-Gly (SEQ ID NO: 12) and the PG is Boc or Fmoc.

9. The method of claim 1, wherein the at least one PG-peptide fragment is D-Phe-Pro-Arg(Pbf)-Pro-Gly-Gly-Gly-Gly, D-Phe-Pro-Arg(Pbf)-Pro-Gly-Gly-Gly, D-Phe-Pro-Arg(Pbf)-Pro-Gly-Gly, or Phe-Pro-Arg(Pbf)-Pro-Gly (SEQ ID NO: 13) and the PG is Boc or Fmoc.

10. The method of claim 1, wherein the at least one PG-peptide fragment is Gly-Gly-Gly-Gly-Asn(Trt)-Gly (SEQ ID NO: 14), Pro-Gly-Gly-Gly-Gly-Asn(Trt)-Gly (SEQ ID NO: 15), Arg(Pbf)-Pro-Gly-Gly-Gly-Gly-Asn(Trt)-Gly (SEQ ID NO: 16), Pro-Arg(Pbf)-Pro-Gly-Gly-Gly-Gly-Asn(Trt)-Gly (SEQ ID NO: 17), or D-Phe-Pro-Arg(Pbf)-Pro-Gly-Gly-Gly-Gly-Asn(Trt)-Gly and the PG is Boc or Fmoc.

11. The method of claim 1, wherein the at least one PG-peptide fragment is Fmoc-Gly-Gly-Gly-Asn(Trt)-Gly (SEQ ID NO: 18).

12. The method of claim 1, wherein the at least one PG-peptide fragment is Fmoc-Gly-Gly-Asn(Trt)-Gly (SEQ ID NO: 19).

13. The method of claim 1, wherein the at least one PG-peptide fragment is Fmoc-Gly-Asn(Trt)-Gly.

14. The method of claim 1, wherein the resin is Wang resin.

15. The method of claim 1, wherein the at least one PG-peptide fragment is Fmoc-Asn(Trt)-Gly and Fmoc-Gly-Gly-Gly-Gly (SEQ ID NO: 20).

16. The method of claim 1, wherein the at least one PG-peptide fragment is Fmoc-Asn(Trt)-Gly and Fmoc-Gly-Gly.

17. The method of claim 1 further comprising:
cleaving the bivalirudin from the resin; and
purifying the cleaved bivalirudin by high pressure liquid chromatography (HPLC).

18. The method of claim 1, wherein the bivalirudin includes an amount of a plus-Gly and/or a minus-Gly impurity of less than about 1.0%.

19. The method of claim 1, wherein the bivalirudin includes an amount of a plus-Gly and/or a minus-Gly impurity of less than about 0.5%.

20. The method of claim 1, wherein the bivalirudin includes an amount of a plus-Gly and/or a minus-Gly impurity of less than about 0.25%.

21. The method of claim 1, wherein the bivalirudin includes an amount of a plus-Gly and/or a minus-Gly impurity of less than about 0.10%.

22. A method for producing bivalirudin using a peptide fragment or peptide fragments on solid phase peptide synthesis (SPPS), comprising:
a) attaching a first protected leucine (Leu) to a resin;
b) de-protecting the protected Leu;
c) reacting a second protected amino acid with the de-protected Leu to form a peptide bond there between,
d) repeating de-protecting and reacting amino acids of 11-19 residues of SEQ ID NO 2 sequentially in a C-terminal to N-terminal direction;
e) de-protecting the protected Asp(OtBu);
f) reacting a protected Asn(Trt)-Gly with the de-protected Asp(OtBu);
g) de-protecting the Asn(Trt);
h) reacting a protected Gly-Gly-Gly-Gly (SEQ ID NO: 3) with the de-protected Asn(Trt);
i) repeating de-protecting and reacting amino acids of 1-4 residues of SEQ ID NO 2, sequentially, and de-protecting the last amino acid residue; and
g) cleaving the bivalirudin from the resin.

23. The method of claim 20, wherein the protecting group is Fmoc or Boc.

24. The method of claim 20, wherein the resin is Wang resin.

25. The method of claim 20, wherein a de-protective agent is used in de-protecting the amino acids, and the de-protective agent comprises an amount of about 3 to 20% of piperidine and an amount of about 0.5 to 10% of bicyclic amidine, based on the total volume thereof.

26. The method of claim 20, wherein a condensing agent is used for reacting the amino acids to form the peptide bonds, and the condensing agent is selected from the group consisting of N,N′-diisopropyl carbodiimide, O-(7-aza-benzotriazole-1-yl)-N,N,N′,N′-tetramethyl uronium hexafluoro phosphate, O-(benzotriazole-1-yl)-N,N,N,N-4-methyl-uronium tetrafluoroborate/N-methyl morpholine, (benzo triazol-1-yl-O)tripyrrolidine phosphonium hexafluorophosphate, 1-hydroxy benzotriazole, and a mixture thereof.

27. The method of claim 20, wherein in the step i), when a protected Arg(Pbf) is reacted, pentafluorophenol is used to condense the protected-Arg(Pbf)-OH with the peptide bound to the resin.

28. The method of claim 20, wherein in the step i), when a protected Arg(HCl) is reacted, pentafluorophenol is used to condense the protected-Arg(Pbf)-OH with the peptide bound to the resin.

29. The method of claim 20, wherein a cleaving agent is used in cleaving the bivalirudin from the resin, and the cleaving agent is trifluoroacetic acid, triisopropyl silane, and water, with a volume ratio thereof 95-60:5-10:5-30.

30. The method of claim 20, wherein the cleaved peptide is precipitated.

31. The method of claim 20, wherein the bivalirudin includes an amount of a plus-Gly and/or a minus-Gly impurity of less than about 1.0%.

32. The method of claim 20, wherein the bivalirudin includes an amount of a plus-Gly and/or a minus-Gly impurity of less than about 0.5%.

33. The method of claim 20, wherein the bivalirudin includes an amount of a plus-Gly and/or a minus-Gly impurity of less than about 0.25%.

34. The method of claim 20, wherein the bivalirudin includes an amount of a plus-Gly and/or a minus-Gly impurity of less than about 0.10%.

35. The method of claim 20, wherein when the PG-peptide fragments includes at least two of Gly-Gly, step h) further comprises:
h-1) reacting a first protected Gly-Gly with the de-protected Asn(Trt);
h-2) de-protecting the Gly,
h-3) reacting a second protected Gly-Gly with the de-protected Gly.

36. A pharmaceutical composition that comprises bivalirudin produced by the method of claim 1.

37. The pharmaceutical composition of claim 34, wherein the bivalirudin includes an impurity of plus-Gly and/or minus-Gly less than about 1.0%.
